(19) United States
(12) Patent Application Publication
(10) Pub. No.: US 2005/0070870 A1
(43) Pub. Date: Mar. 31, 2005

(54) Secret sani-system

(76) Inventor: Sharonda Covington Hayes, San Pedro, CA (US)

Correspondence Address: Sharonda Covington Hayes, 1600 West 27th Street, San Pedro, CA, 90732, US

(21) Appl. No.: 10/672,555

(22) Filed: Sep. 29, 2003

(51) Int. Cl. A61F 13/15; A61F 13/20

(52) U.S. Cl. 604/385.13

(57) ABSTRACT

A sanitary Napkin is described in its usual form. The body of which is planar with two elongate edges and two symmetrical curvilinear ends. The body is composed of an outer impermeable layer, and absorbent core, and a bottom adhesive layer which is affixed to the undergarment of the wearer. The present invention incorporates an internal covering which is pulled from the inside of the sanitary napkin to the outside for disposal of the used sanitary napkin.

### FIELD OF INVENTION

The present invention relates to absorbent articles, in particular sanitary napkins and incontinence pads.

### BACKGROUND OF THE INVENTION

The primary foci of absorbent article, namely sanitary napkins are the efficacy of absorbency or odor control. Another area of consideration is disposal.

Currently, sanitary napkins are disposed of by either tossing the used napkin uncovered, into a receptacle or by folding the used napkin into the plastic covering in which the napkin was originally packaged.

Generally, used sanitary napkins are wrapped in the plastic wrapper in which they came, newspaper (or some other paper) or are just disposed of unwrapped.

Using newspapers or other wrappings is inconvenient. Hence, used sanitary napkins are disposed of unwrapped. This poses a problem of poor sanitation. Prior art poses a sanitary napkin with improved absorbency and/or with odor control. Other prior arts propose better leakage control and transparent fixation flaps.

There remains the problem of inconvenient disposal.

Neither these nor other prior arts address this problem. Consequently, there still exists a need for a more convenient and sanitary method of disposal of used sanitary napkins or incontinence devices.

### SUMMARY

The present invention consists of a sanitary napkin with an intrinsic covering system for disposal of used sanitary napkins.

### OBJECTS AND ADVANTAGES

This invention provides an intrinsic and compact way of covering used sanitary napkins for disposal.

This is accomplished via an internal covering. This covering is nestled inside the sanitary or (Urinary incontinence device). Attached to the covering is a string that leads to the outside of the napkin. Pulling the string brings the covering from inside the napkin to the outside—for the purpose of covering the used napkin.

With this intrinsic system of covering, the used sanitary napkin can be covered before disposal and the problem of disposing used uncovered sanitary napkins is solved. Also, there is no longer a need to find newspaper or other coverings for the used sanitary napkin.

### BRIEF DESCRIPTION OF THE DRAWINGS

The present invention will now be described in greater detail with reference to an embodiment represented in the drawings. The figures show:

FIG. 1—A top cutaway view of the present invention;

FIG. 2—A top view of the present invention;

FIG. 3—The embodiment for disposal;

FIGS. 4 and 4a—The embodiment pre and post package for disposal.

### DETAILED DESCRIPTION OF THE INVENTION

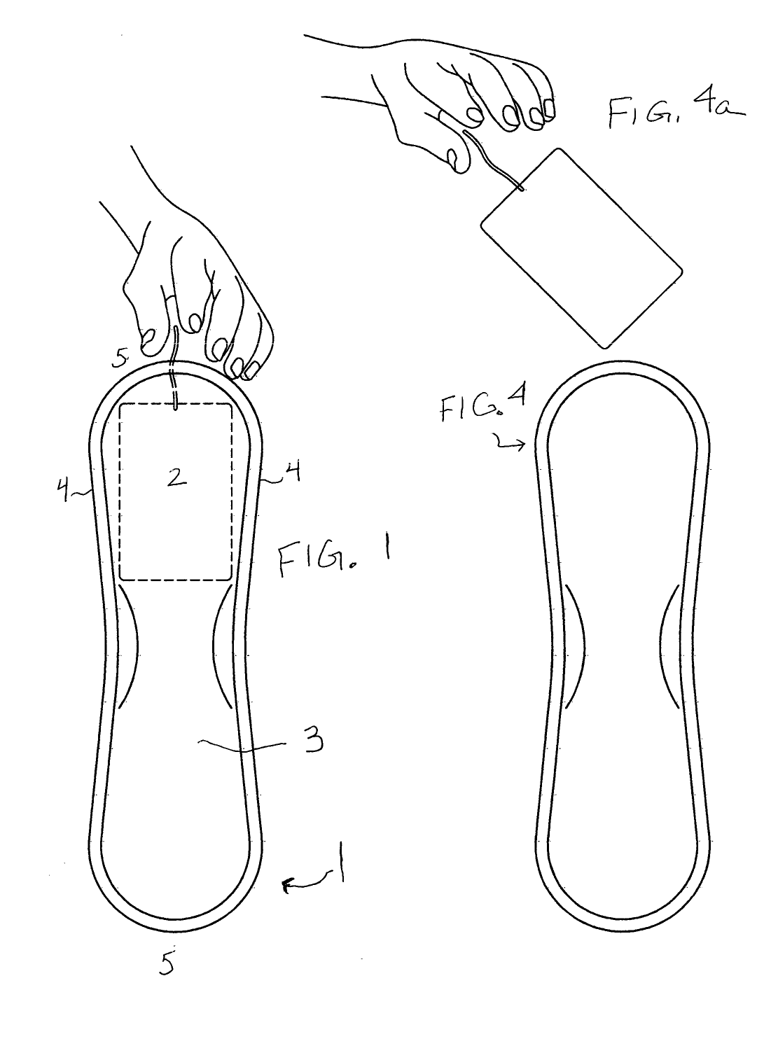

According to a preferred embodiment, and as can be seen from FIGS. 1-4, the sanitary napkin 1 of the present invention if composed of a planar and elongate absorbent main body 3, and two longitudinal side edges, 4. These edges are rectilinear with two transverse curvilinear ends. The main body is composed of a liquid permeable cover layer, which is made to be in contact with the wearer for the purpose of absorbing menstrual flow. Under the absorbent layer is an impermeable layer, which prevents absorbed liquid from leaking out of the sanitary napkin. Lastly, there is an outer layer, which is comprised of adhesive adapted to contact and affix to the user's undergarment. None of these layers are demonstrated. This is the usual embodiment of the sanitary napkin.

The sanitary napkin 1 further comprises an inner material 2 used to cover the used sanitary for disposal. This material is nestled below the impermeable layer and above the outer adhesive layer. This material is essentially a plastic bag with an attached string.

In order to use the sanitary napkin, the string is pulled which brings the bag 2 from inside the sanitary napkin to the outside while still attached to the sanitary napkin.

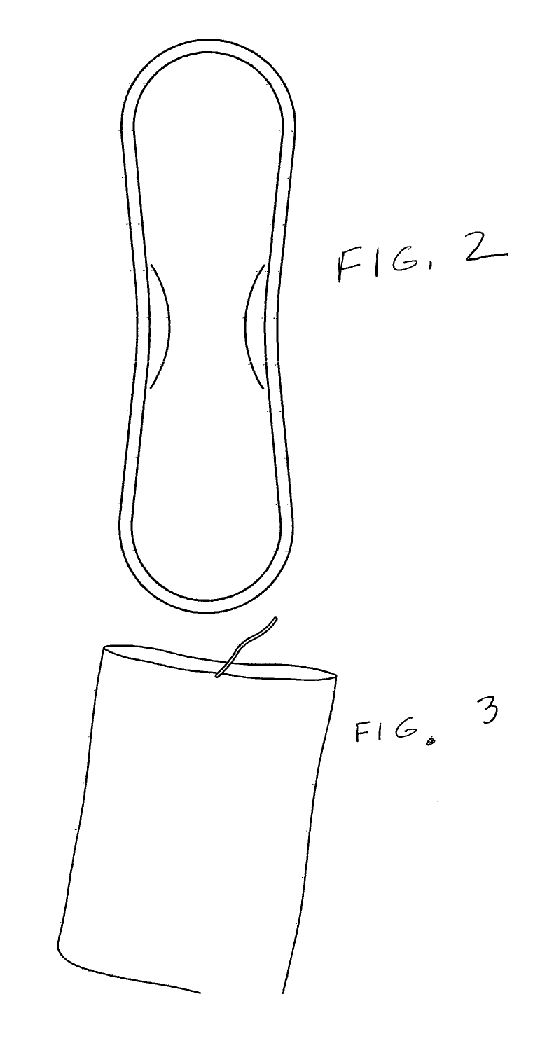

The used sanitary napping is then folded inside the bag for disposal. FIG. 3 shows the embodiment for disposal. FIG. 4 and FIG. 4a shows the embodiment of the sanitary napkin pre and post package respectively for disposal.

The material of the inner bag 2 of FIG. 1 is not limited to plastic. Also, the bag may be placed above or below the impermeable layer of the sanitary napkin. The bag may be placed toward the front, middle, or back of the sanitary napkin. Similarly, the inner bag may be placed in other devices, namely incontinence pads.

A preferred embodiment has been described; but the scope of the present invention may include other variations, and is limited only by the contents of the aforementioned claims, the possible equivalents being included therein.

1. An absorbent article which is characterized an intrinsic covering system for disposal.
An absorbent article according to claim 1 wherein said intrinsic covering is made of a polyethylene material, plastic, or some other material, and is situated inside the absorbent layer and is attached to a string found outside of said absorbent article, whereby pulling the string releases said intrinsic covering.
An absorbent article according to claim 1 wherein said absorbent article is a sanitary napkin or urine/feces incontinence device.
